(19) United States
(12) Patent Application Publication
(10) Pub. No.: US 2026/0262929 A1
(43) Pub. Date: Sep. 10, 2026

(54) Video Laryngoscopy Apparatuses and Methods for Using Same

(71) Applicant: The Regents of the University of California, Oakland, CA (US)

(72) Inventors: Waylan Wong, Orange, CA (US); Gabriel Punsalan, Orange, CA (US)

(73) Assignee: The Regents of the University of California, Oakland, CA (US)

(21) Appl. No.: 19/362,387

(22) Filed: Oct. 18, 2025

Related U.S. Application Data
(63) Continuation-in-part of application No. 17/586,573, filed on Jan. 27, 2022, now Pat. No. 12,458,219.
(60) Provisional application No. 63/142,444, filed on Jan. 27, 2021.

(51) Int. Cl. A61B 1/267; A61B 1/00; A61B 1/015; A61B 1/018; A61B 1/05; A61B 1/12

(52) U.S. Cl. CPC A61B 1/267; A61B 1/00091; A61B 1/00103; A61B 1/015; A61B 1/018; A61B 1/05; A61B 1/126

(57) ABSTRACT

In one embodiment, video laryngoscopy apparatus includes a blade configured for insertion through an oral cavity of a patient, a clear viewing window provided on the blade that enables the patient's airway to be imaged when a video device is received by the video laryngoscopy apparatus, and a fluid nozzle configured to eject a jet of fluid across the viewing window to drive the material from an outer surface of the viewing window. In another embodiment, a video laryngoscopy apparatus includes one or more flexible tubes that are removably attached to the apparatus that can be partially or fully separated from the apparatus to enable the tubes to be used to advance one or more other medical instruments into the patient's airway.

### CROSS-REFERENCE TO RELATED APPLICATION

This application is a continuation-in-part and claims the benefit of co-pending U.S. Non-Provisional patent application Ser. No. 17/586,573, filed Jan. 27, 2022, which claims the benefit of U.S. Provisional Application No. 63/142,444, filed Jan. 27, 2021, both of which are hereby incorporated by reference herein in their entireties.

### NOTICE OF GOVERNMENT-SPONSORED RESEARCH

This invention was made with Government support under grant contract number R44HL164325 awarded by the National Institutes of Health (NIH). The Government has certain rights in the invention.

### BACKGROUND

Video laryngoscopy is often used during emergency breathing tube placement to save lives. However, materials from within the body, such as airway secretions, gastric fluid, blood, and/or solids, such as regurgitated food, can cover the viewing window of the laryngoscope camera and, therefore, obscure the view of the airway. This can lead to breathing tubes being misplaced within the esophagus instead of the trachea. Because the patient may not be breathing at the time, the attending medical professional is under extreme time pressure to place the breathing tube within the patient's airway to restore respiration and prevent aspiration. If he or she cannot achieve this in a timely manner, the result can be hypoxemia, acidosis, and even death.

It is further noted that video laryngoscopes can comprise features that enable introduction of other medical instruments into the airway. Unfortunately, such features can add bulk to the laryngoscope that render it more difficult to use and/or that can make undesired complications more likely to occur.

In view of the above facts, it can be appreciated that it would be desirable to have a video laryngoscopy apparatus that includes means for clearing material from a viewing window of the apparatus and/or that includes features that facilitate the introduction of other medical instruments into the patient airway without unduly increasing the bulk of the apparatus.

### BRIEF DESCRIPTION OF THE DRAWINGS

The present disclosure may be better understood with reference to the following figures. Matching reference numerals designate corresponding parts throughout the figures, which are not necessarily drawn to scale.

FIG. 1 is a first perspective view of a first embodiment of a video laryngoscopy apparatus.

FIG. 2 is a top view of the video laryngoscopy apparatus of FIG. 1.

FIG. 3 is a front view of the video laryngoscopy apparatus of FIG. 1.

FIG. 4 is a first perspective detail view of a distal end of a blade of the video laryngoscopy apparatus of FIG. 1.

FIG. 5 is a second perspective detail view of the distal end of the blade of the video laryngoscopy apparatus of FIG. 1.

FIG. 6 is a detail view of an underside of the distal end of the blade of the video laryngoscopy apparatus of FIG. 1.

FIG. 7 is a first perspective view of a second embodiment of a video laryngoscopy apparatus.

FIG. 8 is a second perspective view of the video laryngoscopy apparatus of FIG. 7.

FIG. 9 is a perspective detail view of a proximal end of a handle of the video laryngoscopy apparatus of FIG. 7.

FIG. 10 is a first perspective detail view of a distal end of a blade of the video laryngoscopy apparatus of FIG. 7.

FIG. 11 is a second perspective detail view of the distal end of the blade of the video laryngoscopy apparatus of FIG. 7.

FIG. 12 is a first perspective view of a third embodiment of a video laryngoscopy apparatus.

FIG. 13 is a second perspective view of the video laryngoscopy apparatus of FIG. 12.

FIG. 14 is a first perspective detail view of a distal end of a blade of the video laryngoscopy apparatus of FIG. 12.

FIG. 15 is a second perspective detail view of the distal end of the blade of the video laryngoscopy apparatus of FIG. 12.

### DETAILED DESCRIPTION

As described above, it would be desirable to have a video laryngoscopy apparatus that includes means for clearing material from a viewing window of the apparatus and/or that includes features that facilitate the introduction of other medical instruments into the patient airway without unduly increasing the bulk of the apparatus. Examples of such apparatuses are disclosed herein.

In one embodiment, a video laryngoscopy apparatus comprises a disposable video laryngoscopy sleeve configured to receive a video device, such as a video baton or video laryngoscope. The apparatus comprises a blade that is configured for insertion into a patient airway and that includes a clear viewing window through which images of the airway can be captured by a camera of the video device. The apparatus further comprises means for clearing material from the viewing window that utilizes fluid to drive the material from an outer surface of the window. In addition, the apparatus comprises means for removing the fluid and the cleared material from the airway.

In another embodiment, the video laryngoscopy apparatus further comprises one or more flexible tubes secured to the apparatus with means for removably securing the tubes. In some embodiments, a first tube can be used to supply the fluid for clearing material from the viewing window and a second tube can be used to apply suction to remove the fluid and cleared material from the airway. In some embodiments, the second tube further can be used as working channel through which another medical instrument can be passed and advanced into the airway and/or can be partially or fully separated from the remainder of the apparatus so that the tube can be independently used for another purposes, such as introducing an endotracheal tube into the trachea.

In the following disclosure, various particular embodiments are described. It is to be understood that those embodiments are example implementations of the disclosed inventions and that alternative embodiments are possible. Such alternative embodiments include hybrid embodiments that include features from different disclosed embodiments. All such embodiments are intended to fall within the scope of this disclosure.

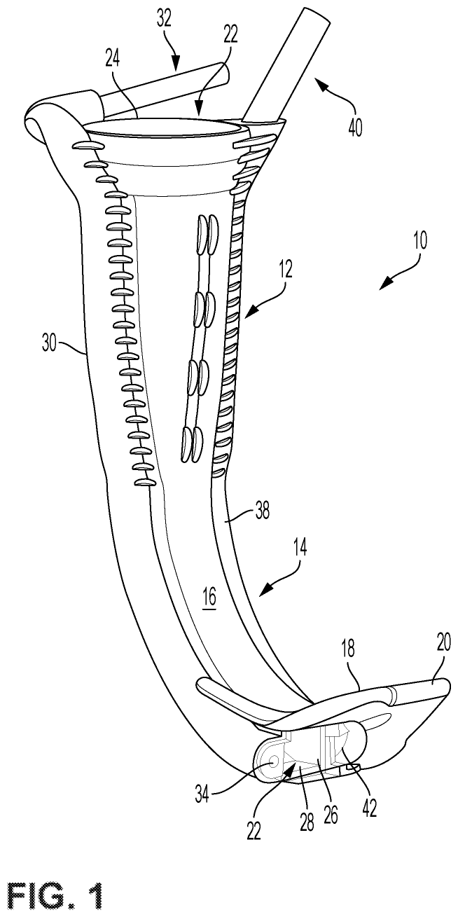

FIG. 1 illustrates a first example embodiment of a video laryngoscopy apparatus 10 that includes means for clearing material from a viewing window of the apparatus. The example apparatus 10 of FIG. 1 comprises a disposable video laryngoscopy sleeve or sheath that is configured to receive a video device, such as a video baton or video laryngoscope. For the remainder of the discussion of the video laryngoscopy apparatus 10, the apparatus will be referred to as a sleeve 10. As will be apparent from the disclosure that follows, when a video device is received by the sleeve 10, the resulting combination can be referred to and used as a video laryngoscope.

The sleeve 10 generally includes a handle 12 that is configured to be gripped by an operator and an elongated, curved blade 14 that is configured for insertion through the mouth and oral cavity, and into the throat for the purpose of intubating a patient. In some embodiments, the handle 12 and blade 14 are unitarily constructed of a single piece of inexpensive, rigid or semi-rigid material, such as a polymer material, so the sleeve 10 can be inexpensively produced and, therefore, is suitable for one-time use and disposability. As is apparent from FIG. 1, the blade 14 includes a relatively thick proximal portion 16 that extends from the handle 12 and a relatively thin distal portion 18 that extends from the proximal portion. The distal portion 18 forms a distal tip 20 of the blade 14 that, in some embodiments, can be placed within the vallecula between the base of the tongue and the front of the epiglottis.

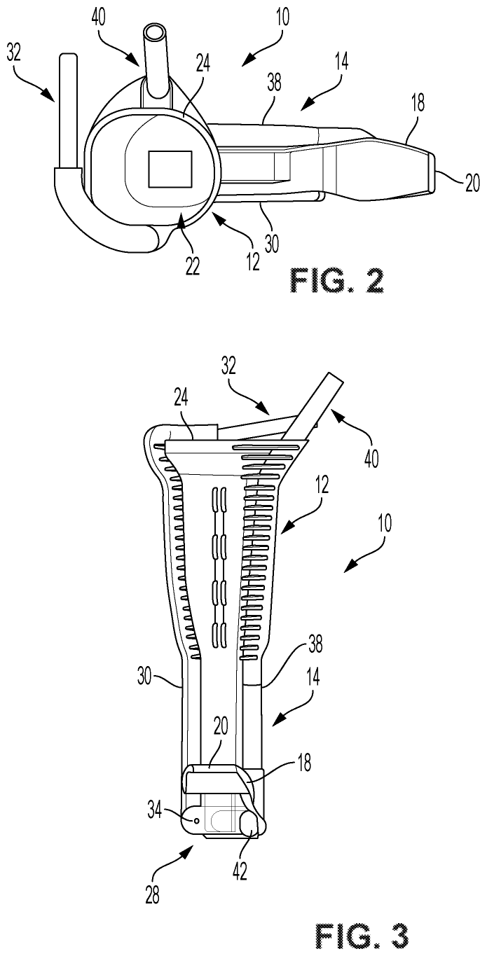

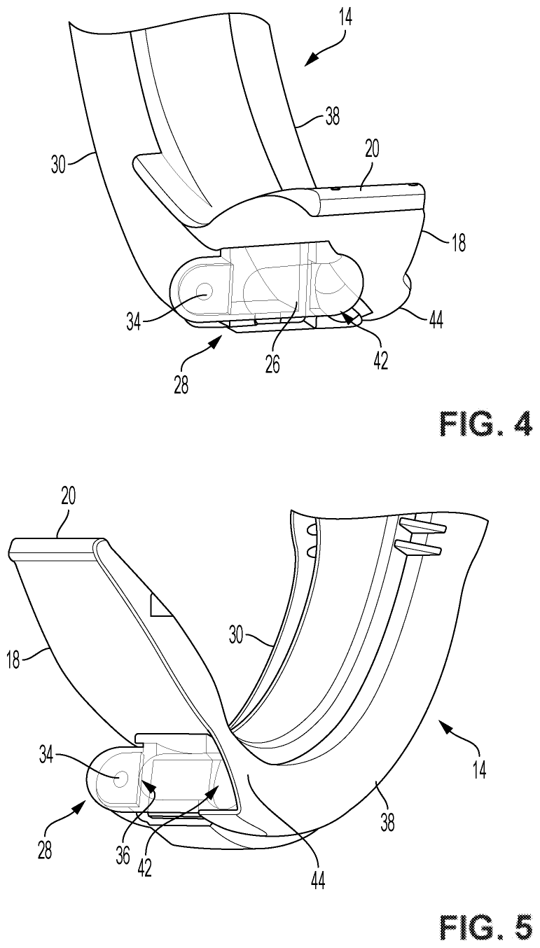

Extending through the handle 12 and the proximal portion 16 of the blade 14 is a relatively large, central, first channel 22 that is configured to receive the video device. As such, the first channel 22 also may be referred to as the video device channel. In some embodiments, the first channel 22 is specifically configured to receive, through a proximal opening 24 of the channel formed at the proximal end of the handle 12, a video baton (not shown) that comprises a body, a camera, and a shaft that extends between the body and camera. By way of example, a body of the baton can be placed within a proximal portion of the first channel 22 formed by the handle 12, the shaft can be placed in a distal portion of the channel, and the camera can be positioned adjacent to a distal opening 26 of the channel formed by the blade 14. As shown most clearly in FIGS. 2 and 4, the first channel 22 can, in some embodiments, have a generally rectangular cross-section, at least within the proximal portion 16 of the blade 14.

It is noted that, although a video baton has been identified as an example video device that can be received by and used with the sleeve 10, other video devices could be used with the sleeve. Indeed, substantially any video device having a camera that can be positioned within the distal portion of the first channel 22 against or adjacent to an integrated viewing window of the sleeve 10 can be used. Furthermore, it is noted that the video device need not include a camera that is positioned within the blade 14 or even the handle 12 of the sleeve 10. For example, the video device can comprise one or more optical waveguides, such as optical fibers, that can be inserted through the first channel 22 in a manner in which their distal ends or a lens associated therewith is/are positioned in contact with or adjacent to the viewing window. In such a case, the one or more optical waveguides can transmit images of the patient airway to a camera that is either positioned within or outside of the sleeve 10.

With further reference to FIG. 1, the sleeve 10 also includes a second channel 30 that forms part of and extends along a first lateral side (the right side from the perspective of the operator) of the handle 12 and the proximal portion 16 of the blade 14. The second channel 30 originates with a first proximal tube 32 that extends from the proximal end of the handle 12 and ends with a distal opening 34 that is positioned at the distal end of the proximal portion 16 of the blade 14 laterally adjacent to the distal opening 26 of the first channel 22. As described below, the second channel 30 can be used to supply fluid to the distal end of the proximal portion 16 of the blade 14 for use in clearing material from a viewing window of the sleeve 10, which is described below. As such, the second channel 30 also may be referred to as a fluid supply channel and its distal opening 34 also may be referred to as an outlet opening. The fluid that is supplied by the second channel 30 can comprise a gas, a liquid, or a combination of the two. Example gases include air and oxygen. Example liquids include water, saline, as well as solutions that contain one or more beneficial ingredients, such as one or more medications.

Provided on a second lateral side (the left side from the perspective of the operator) of the handle 12 and the proximal portion 16 of the blade opposite to the second channel 30 is a third channel 38. The third channel 38 originates with a second proximal tube 40 that extends from the proximal end of the handle 12 and ends with a distal opening 42 that is also positioned at the distal end of the proximal portion 16 of the blade 14 laterally adjacent to the distal opening 26 of the first channel 22. As described below, the third channel 38 can be used for a variety of purposes. For example, the third channel 38 can be used as a suction channel through which at least some of the fluid supplied by the second channel 30 and the material cleared by that fluid can be removed from the patient's throat. For that reason, the third channel 38 also may be referred to as a suction channel and its distal opening 42 also may be referred to as an inlet opening. As another example, the third channel 38 can be used as a working channel through which other instruments can be passed to perform some function within the patient's throat. Because the third channel 38 can be used for a variety of purposes, the third channel also may be referred to as an auxiliary channel.

Mounted to the distal end of the proximal portion 16 on an underside of the blade 14 is an end piece 28 that is made of clear material, such as a clear polymer material like polycarbonate. As is most clearly apparent from FIGS. 4-6, a central portion of the end piece 28 covers the distal opening 26 of the first channel 22 in which the video device is placed, and a lateral portion of the end piece covers the distal opening 34 of the second channel 30. Given that the central portion of the end piece 28 overlies the distal opening 26 of the first channel 22, the central portion forms a clear viewing window through which images and/or video (video being a series of sequential images) can be captured by the camera of the video device that is used with the sleeve 10. In addition, the end piece 28 seals the distal opening 26 of the first channel 22 so that material, such as liquids and/or solids from within the patient, cannot enter the first channel.

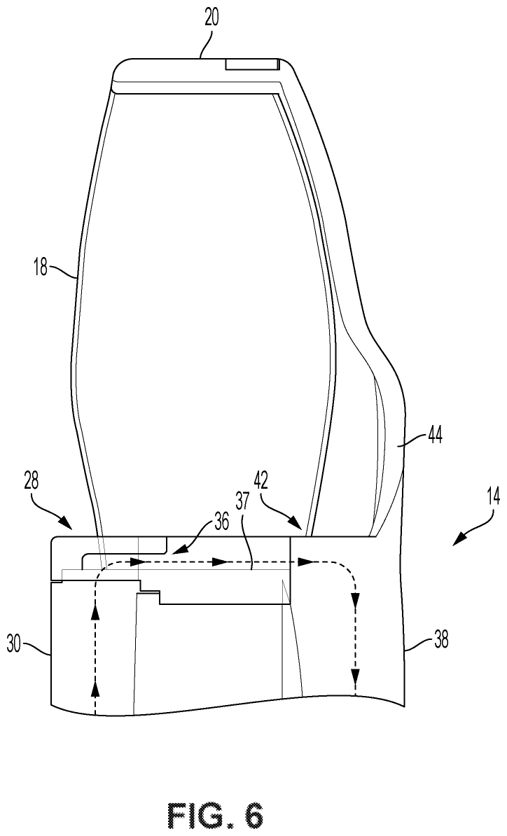

While the central portion of the end piece 28 forms the viewing window, the lateral portion of the end piece forms a fluid nozzle 36 from which fluid can be ejected to clear material from the viewing window. Accordingly, the end piece 28 integrates both the viewing window and the fluid nozzle 36, which are both unitarily formed from a single piece of material from which the end piece is made. The fluid nozzle 36 is in fluid communication with the distal opening 34 of the second channel 30 and, therefore, is configured to receive the fluid that is supplied to the nozzle by the second channel. When supplied with that fluid at a sufficient pressure, the fluid nozzle 36 ejects a high-velocity jet of the fluid transversely across a planar outer surface 37 (see FIG. 6) of the viewing window.

The dashed line in FIG. 6 identifies the path of the fluid as it travels through the second channel 30, through and out from the fluid nozzle 36, and across the outer surface 37 of the viewing window. As can be appreciated from that path, the ejected jet of fluid travels in a direction that is transverse and generally parallel to the outer surface 37 of the viewing window so that the fluid can drive any material that has accumulated on the surface from a first lateral side (e.g., the right side) of the window to a second, opposite lateral side (e.g., the left side) of the window to ensure the material does not obscure the view of the patient airway. Notably, as the ejected fluid and whatever material it clears is driven across the viewing window, it travels toward the distal opening 42 of the third channel 38, which can be used to remove the fluid and material from the airway.

Example constructions of the sleeve 10 having been described above, use of the sleeve will next be discussed. In a first example use case, the third channel 38 is used as a suction channel. In such a case, a suction source (not shown) can be connected to the second proximal tube 40 so that fluid and material cleared from the viewing window can be removed. Accordingly, the second channel 30, the fluid nozzle 36, and the third channel 38 can be used together to clear and remove material that could otherwise obstruct the operator's view of the patient airway. Referring to FIGS. 4-6, the blade 14 further can include a lateral flange 44 adjacent to the distal opening 42 of the third channel 38 on the second lateral side (e.g., left side) of the blade. As is apparent from those figures, the flange 44 extends outward from the distal end of the proximal portion 16 of the blade 14 and downward from at least part of the distal portion 18 of the blade in an orientation in which it is generally perpendicular to the outer surface 37 of the viewing window and the direction the ejected jet of fluid travels. With that configuration and placement, the flange 44 can act as a backstop for the ejected fluid and cleared material to assist in the collection and removal of the fluid and material via the third channel 38.

It is further noted that, when used to provide suction, the third channel 38 of the sleeve 10 can also be used to remove airborne particles from within the patient airway. Such particles can, for example, comprise aerosolized virus particles or other bio-aerosol contaminants. The particles captured and removed from the airway via the third channel 38 can be contained within a suitable vessel, such as a suction cannister, to prevent contamination of the surrounding area and/or infection of healthcare providers within the area. In further embodiments, the sleeve 10 can include one or more integrated features configured to destroy or otherwise inactivate such contaminants.

In a second example use case, the third channel 38 can be used as a working channel through which other medical instruments (not shown) can be passed and advanced into the patient airway. For example, a suction catheter can be passed through the third channel 38 and used to clear the ejected fluid and patient material in similar manner to when the channel is used as a suction channel. When a suction catheter is used, however, it can be extended beyond the distal end of the proximal portion 16 of the blade 14 to remove material that exists beyond the distal opening 42 of the third channel 38. As another example, a guidewire can be passed through the third channel 38 and, when left within the airway after the sleeve 10 has been removed, can be used to introduce another medical instrument, such as an endotracheal tube, into the airway or the trachea. In such a case, the sleeve 10 functions as an introducer.

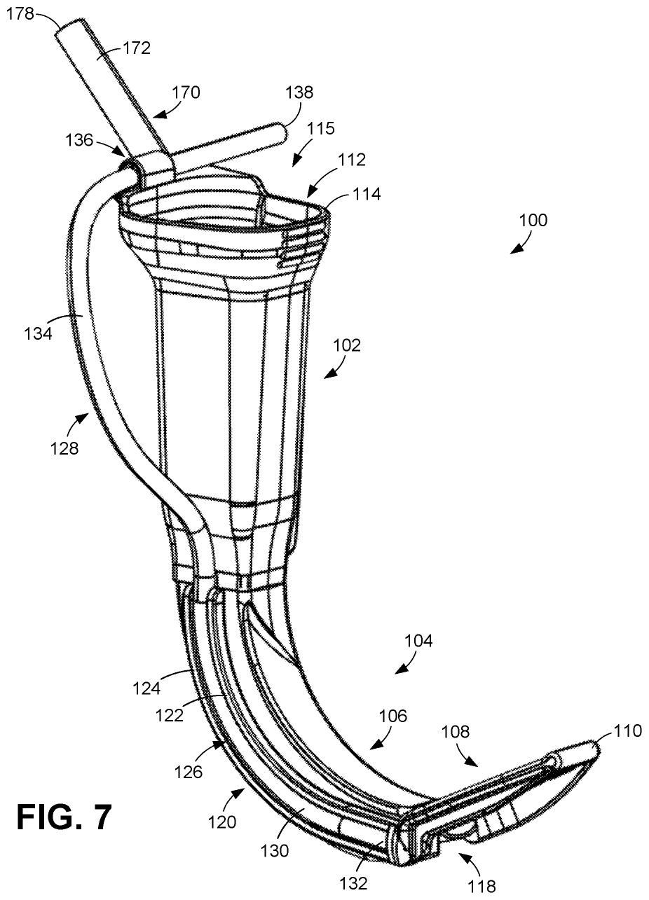

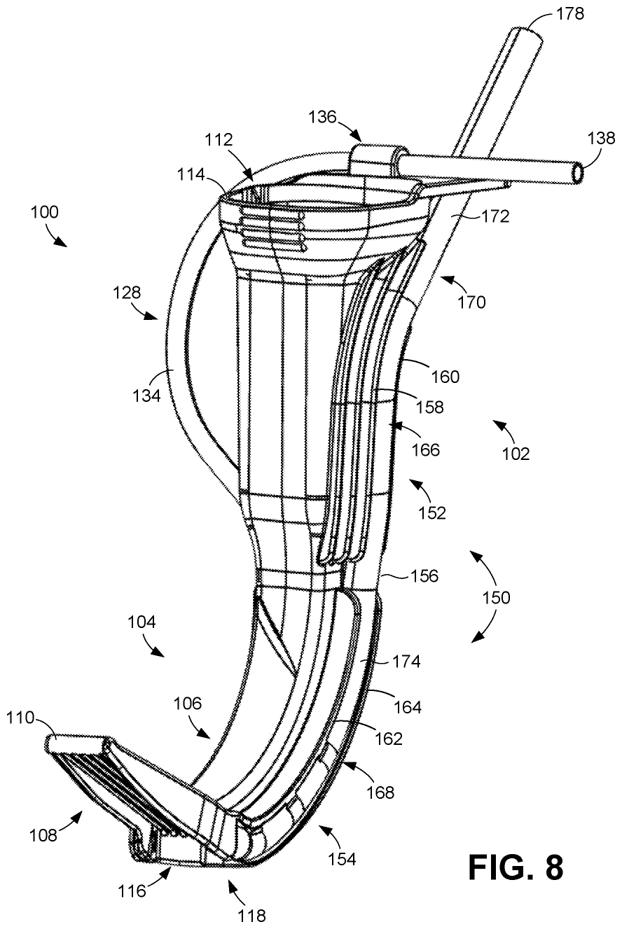

FIGS. 7 and 8 illustrate a second example embodiment of a video laryngoscopy apparatus 100 that includes means for clearing material from a viewing window of the apparatus. As with the apparatus 10 of FIG. 1, the apparatus 100 shown in FIGS. 7 and 8 comprises a disposable video laryngoscopy sleeve or sheath that is configured to receive a video device, such as a video baton or video laryngoscope. For the remainder of the discussion of the video laryngoscopy apparatus 100, the apparatus will be referred to as a sleeve 100. As will be apparent from the disclosure that follows, when a video device is received by the sleeve 100, the resulting combination can be referred to and used as a video laryngoscope.

The sleeve 100 shares several similarities with the sleeve 10 of FIG. 1. Accordingly, the sleeve 100 generally includes a handle 102 that is configured to be gripped by an operator and an elongated, curved blade 104 that is configured for insertion through the mouth and oral cavity, and into the throat for the purpose of intubating a patient. In some embodiments, the entire sleeve 100, including the handle 102, blade 104, and their various components, are unitarily constructed of a single piece of an inexpensive, rigid or semi-rigid, clear material, such as a polymer material. For example, the entire sleeve 100 can be made of a clear polymer material, such as polycarbonate. Although the completed sleeve 100 has a unitary construction, it is noted that one or more portions of the sleeve can be separately formed and later joined together during the manufacturing process to form the unitary sleeve. For example, in some embodiments, the blade 104 can be injection molded from the polymer material and the handle 102 can then be over-molded onto the blade, resulting in a finished sleeve 100 formed by a single piece of polymer material.

Constructing the sleeve 100 from an inexpensive, clear material provides advantages over constructing the sleeve using other materials. As described below, the sleeve 100 includes an integrated viewing window that is unitarily formed with the blade 104 through which images of a patient airway can be captured. By making the sleeve 100, and therefore the blade 104, out of a clear material, the viewing window will likewise be clear, obviating the need to secure a separately manufactured window to the blade, simplifying manufacturing, and reducing manufacturing costs. The use of an inexpensive material for the construction of the sleeve 100 further reduces manufacturing costs and also enables disposability of the sleeve.

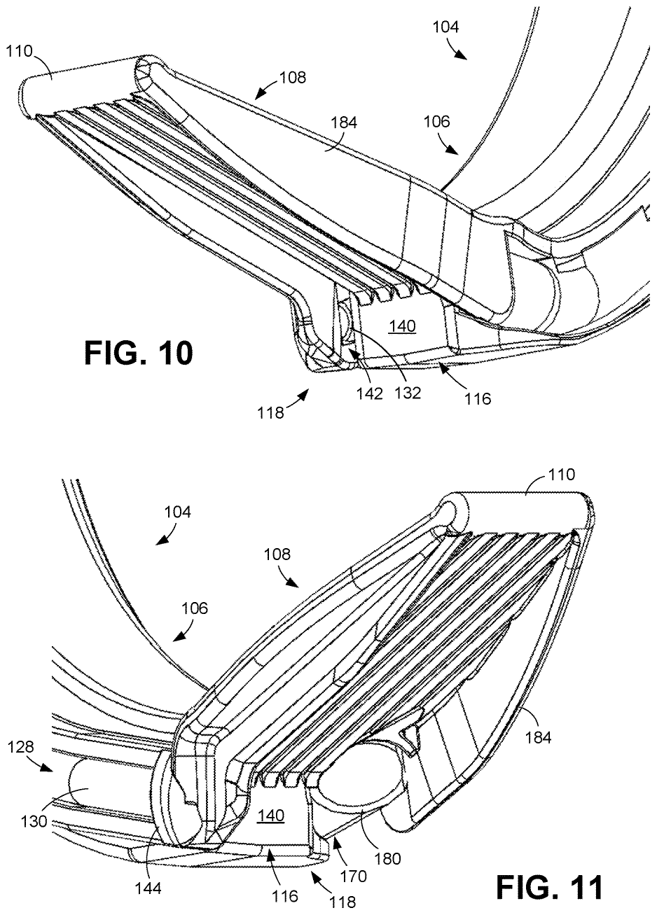

As is apparent from FIGS. 7 and 8 (as well as FIGS. 10 and 11), the blade 104 includes a relatively thick proximal portion 106 that extends from the handle 102 and a relatively thin distal portion 108 that extends from the proximal portion. The distal portion 108 forms a distal tip 110 of the blade 104. Extending through the handle 102 and the proximal portion 106 of the blade 104 is a relatively large, central, first channel 112 that is configured to receive the video device, such as a video baton. As such, the first channel 112 also may be referred to as the video device channel. In some embodiments, the first channel 112 is specifically configured to receive, through a proximal opening 114 of the channel formed at a proximal end 115 of the handle 102, a video device comprising a camera that can be positioned adjacent to a clear viewing window 116 that terminates the channel at a distal end 118 of the proximal portion 106 of the blade 104, as shown in FIG. 8. As is apparent from that figure, the viewing window 116 can have a generally rectangular cross-section. In similar manner, the portion of the first channel 112 that extends through the proximal portion 106 of the blade 104 can also have a generally rectangular cross-section.

As was noted above in relation to the sleeve 10, although a video baton has been identified as an example video device that can be received by and used with the sleeve 100, substantially any video device having a camera that can be positioned within the distal portion of the first channel 112 against or adjacent to a viewing window 116 of the sleeve could be used. Furthermore, the video device need not include a camera that is positioned within the blade 104 or even the handle 102 of the video laryngoscopy apparatus 100. For example, the video device can comprise one or more optical waveguides, such as optical fibers, that can be inserted through the first channel 112 and used to transmit images of the patient airway to a camera of the device that is either positioned within or outside of the sleeve 100.

With reference to FIG. 7, the sleeve 100 also includes a second channel 120 that forms part of and extends along a first lateral side (the right side from the perspective of the operator) of the proximal portion 106 of the blade 104. Unlike the second channel 30 of the sleeve 10, however, the second channel 120 of the sleeve 100 does not extend along the handle 102 of the sleeve. This difference reduces the width of the handle 102 and may make the sleeve 100 more ergonomic for some operators, thereby making the sleeve easier to grip and manipulate. Also unlike the second channel 30, the second channel 120 is an elongated, open, external channel that extends along the first lateral side of the sleeve 100 and, more particularly, the proximal portion 106 of the blade 104. The base of the second channel 120 is defined by the outer surface of the proximal portion 106 of the blade 104, and the sides of the channel are defined by two spaced, parallel, elongated flanges 122 and 124 that extend generally perpendicularly outward from the outer surface. With that configuration, the outer surface and the flanges 122, 124 define an interior space 126 configured to receive and hold a detachable first tube 128, and the second channel 120 may be described as a C- or U-shaped channel because it has a C- or U-shaped cross-section.

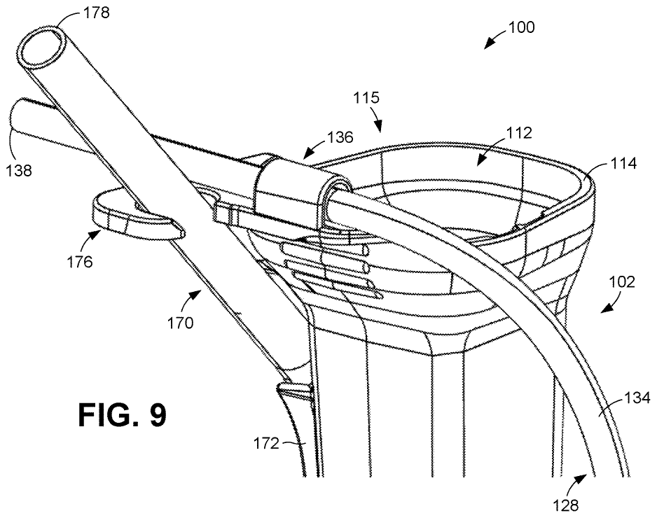

As shown in FIG. 7, the detachable first tube 128 can be positioned within the interior space 126 defined in part by the flanges 122, 124 so that a distal portion 130 of the first tube occupies the entire length of the second channel 120 and a distal opening 132 of the first tube is positioned at a distal end of the second channel 120, which coincides with the distal end 118 of the proximal portion 106 of the blade 104. As is also shown in FIG. 7, a proximal portion 134 of the first tube 128 that does not fit within the second channel 120 is free from the sleeve 100 and extends upward along the handle 102 to a tube coupler 136 that is provided at a rear edge of the proximal end 115 of the handle. As is shown most clearly in FIG. 9, the first tube 128 passes through the coupler 136, which secures the tube to the handle 102, and a small length of the tube extends out beyond the coupler to provide access to a proximal opening 138 of a proximal end of the tube to which a fluid source can be connected. As described below, fluid from the fluid source can be supplied through an inner lumen of the first tube 128 to the distal end 118 of the proximal portion 106 of the blade 104 for use in clearing material from the viewing window 116. In view of that, the first tube 128 also may be referred to as a fluid supply tube, its distal opening 132 also may be referred to as an outlet opening, and the second channel 120 also may be referred to as a supply channel.

The first tube 128 is flexible and can have an outer dimension (e.g., outer diameter) that is slightly greater than the width of the second channel 120 such that the tube is secured within the channel by compressive force and friction provided by the flanges 122, 124. Although that compressive force and friction are enough to maintain the first tube 128 in place within the second channel 120, the tube easily can be removed from the channel by the operator by simply pulling on the tube with enough force to overcome the compressive force and friction to separate the tube from the channel. In view of that, the second channel 120 also may be referred to as means for releasably securing the first tube 128. In other embodiments, one or more releasable locking elements (additional means for releasably securing) can be provided on the proximal portion 106 of the blade 104 to ensure the first tube 128 is securely held within the second channel 120 unless and until the operator wishes to remove the tube from the channel. Such locking elements can, for example, comprise one or more quick-release latches, magnetic couplings, bayonet-type twist-locks, frangible joints, shear-notches, or some combination of two or more of those elements.

FIG. 10 illustrates an underside of a distal end of the blade 104 and, more particularly, the distal end 118 of the proximal portion 106 of the blade. As shown in the figure, the viewing window 116 includes a planar outer surface 140 and forms part of the distal end 118 of the proximal portion 106 of the blade 104 because the viewing window is integrated and unitarily formed with the remainder of the blade. On a first lateral side (the right side from the perspective of the operator) of the viewing window 116 is a fluid passage 142 through which fluid ejected from the distal opening 132 of the tube can flow for the purpose of removing material from the outer surface 140 of the viewing window. The fluid ejected by the first tube 128 is forced to pass through the fluid passage 142 due to the provision of an end wall 144 provided at the distal end of the second channel 120, which is most clearly visible in FIG. 11. Specifically, the distal opening 132 of the first tube 128 is positioned in close proximity to the end wall 144 but with a small gap therebetween so that fluid ejected from the tube has no other exit from the second channel 120 or the remainder of the blade 104. Accordingly, the fluid ejected from the first tube 128 can only escape the second channel 120 via the fluid passage 142.

As the end wall 144 is generally perpendicular to the fluid passage 142, the end wall causes a generally 90-degree change of direction of the flow of the fluid so that, instead of traveling in a direction generally parallel to the distal portion 106 of the blade 104, the fluid is forced to travel in a direction that is generally parallel to the outer surface 140 of the viewing window 116, similar to the jet of fluid ejected by the nozzle 36 of the sleeve 10. As such, the ejected fluid travels transversely across the viewing window 116 in a direction generally parallel to the outer surface 140 of the viewing window so as to traverse the viewing window from a first lateral side (the right side from the perspective of the operator) to a second lateral side (the left side from the perspective of the operator) of the window. In some embodiments, the fluid passage 142 is sized and configured to form a nozzle from which a jet of fluid, such as a gas or a liquid, is ejected with sufficient force to clear substantially any material that has accumulated on the outer surface 140 of the viewing window 116 to ensure a clear view of the patient airway.

With reference back to FIG. 8, the sleeve 100 further includes a third channel 150 that forms part of and extends along a second lateral side (the left side from the perspective of the operator) of the sleeve 100. In the illustrated example, the third channel 150 is divided into two parts: a proximal portion 152 that extends along the handle 102, and a distal portion 154 that, like the second channel 120, extends along the proximal portion 106 of the blade 104. As is apparent from FIG. 8, the two portions 152, 154 are separated by a small gap 156 positioned at a transition point between the handle 102 and the blade 104.

The third channel 150 is also an elongated, open, external channel. The base of the proximal portion 152 of the third channel 150 is defined by an outer surface of the handle 102, and the base of the distal portion 154 of the channel is defined by the outer surface of the proximal portion 106 of the blade 104. The sides of the third channel 150 are defined by two spaced, parallel, elongated flanges 158 and 160 that extend generally perpendicularly outward from the outer surface of the handle 102 along the proximal portion 152, and by two spaced, parallel, elongated flanges 162 an 164 that extend generally perpendicularly outward from the outer surface of the blade 104 along the distal portion 154. With that configuration, the flanges 158-164 define a proximal interior space 166 along the proximal portion 152 of the third channel 150, and a distal interior space 168 along the distal portion 154 of the third channel, and the third channel may be described as a C- or U-shaped channel. Like the second channel 120, the proximal and distal portions 152, 154 of the third channel 150 are configured to receive and hold a tube, in this case a detachable second tube 170, but enable the tube to be manually removed by the operator.

As shown in FIG. 8, the detachable second tube 170 can be positioned with both the proximal and distal portions 152, 154 of the third channel 150 so that a proximal portion 172 of the tube occupies the length of the proximal portion of the channel and a distal portion 174 of the tube occupies the length of the distal portion of the channel. With reference to FIG. 9, the proximal portion 172 of the second tube 170 extends from the proximal end of the proximal portion 152 of the third channel 150 to a hook-shaped tube retainer 176 that secures the tube to the handle 102. A short length of the second tube 170 extends beyond the retainer 176 and terminates in a proximal opening 178 provided at a proximal end of the tube that can be connected to a suction source.

As described below, when a suction source is connected to the second tube 170, the second tube can be used to remove at least some of the fluid ejected from the first tube 128 and the material the fluid has removed from the outer surface 140 of the viewing window 116. In particular, the fluid and material can be received by a distal opening 180 (see FIG. 11) of the second tube 170, drawn up through the inner lumen of the tube, and out from the sleeve 100. In such cases, the second tube 170 also may be referred to as a suction tube, the distal opening 180 also may be referred to as an inlet opening, and the third channel 150 also may be referred to as a suction channel. Notably, however, the second tube 170, as well as the third channel 150, can be used for other purposes, as described below.

With reference to FIG. 11, a distal opening 180 of the second tube 170 is positioned at a distal end of the third channel 150, which coincides with the distal end 118 of the proximal portion 106 of the blade 104. As is apparent from FIGS. 10 and 11, the distal opening 180 of the second tube 170 is positioned on a second lateral side (the left side from the perspective of the operator) of the viewing window 116 so that it is positioned on the opposite side of the viewing window from the fluid passage 142. As is apparent from FIG. 10, the distal opening 180 of the second tube 170 can be beveled so as to have an elliptical shape having a long axis that is greater than the diameter of the inner lumen of the tube and generally perpendicular to the viewing window 116. With that configuration and positioning, the distal opening 180 is well suited to draw in and remove liquid ejected from the fluid passage 142 and material the liquid has cleared from the viewing window 116. That is, the fluid ejected from the fluid passage 142 can travel transversely across the viewing window 116 from a first (right) side of the window to a second (left) side of the window in a direction generally parallel to its outer surface 140, and then pass through the distal opening 180 of the second tube 170 so that it can be removed through the tube by suction force.

As with the first tube 128, the second tube 170 is flexible and can be deformed so that, when the tube has an outer dimension (e.g., outer diameter) that is slightly greater than the width of the third channel 150, the tube is secured within the channel by compressive force and friction provided by the flanges 158-164. The second tube 170, however, can be removed from the third channel 150 by simply pulling on the tube with enough force to overcome the compressive force and friction to separate the tube from the channel. In view of that, the third channel 150 also may be referred to as means for releasably securing the second tube 170. As with the second channel 120, one or more releasable locking elements (additional means for releasably securing) can be provided on the sleeve 100 to ensure that the second tube 170 is securely held within the third channel 150 unless and until the operator wishes to remove the tube from the channel. Such locking elements can, for example, comprise one or more quick-release latches, magnetic couplings, bayonet-type twist-locks, frangible joints, shear-notches, or a combination of two or more of those elements.

With further reference to FIGS. 10 and 11, the blade 104 further can include a lateral flange 184 adjacent to the distal opening 180 of the second tube 170. As is apparent from those figures, the flange 184 extends outward from the distal end 118 of the proximal portion 106 of the blade 104 and downward from at least part of the distal portion 108 of the blade in an orientation in which it is generally perpendicular to the outer surface 140 of the viewing window 116. With that configuration and placement, the flange 184 can act as a backstop for the ejected fluid and cleared material that has been driven across the viewing window 116 to assist in the collection and removal of the fluid and material via the second tube 170. It is further noted that, when used to provide suction, the third channel 38 also can be used to remove contaminants, such as aerosolized particles, from the patient airway.

Notably, the second tube 170, as well as the third channel 150 in which the tube is positioned, can be used for purposes in addition or in exception to applying suction to remove the ejected fluid and patient material from the patient airway. For example, the inner lumen of the second tube 170 can be used as a working channel through which other medical instruments can be passed and positioned within the airway and, if applicable, can be used to perform some action therein. Examples of such other medical instruments include guidewires, airway-exchange catheters, suction catheters, forceps, retrieval devices, gastric tubes, cautery probes, diagnostic sensors, and imaging devices.

Further uses of the second tube 170 and/or the third channel 150 are possible because the second tube is an independent component that can be removed from the proximal portion 152 of the third channel 150, the distal portion 154 of the third channel, or both. Notably, such removal can be achieved through lateral separation, in which the second tube 170 is pulled in a generally perpendicular direction away from the sleeve 100 and out of the channel 150, through axial separation in which the second tube is pulled in a generally axial direction along the sleeve that is generally coincident with or parallel to a longitudinal axis of the channel, or some combination thereof. In cases in which the second tube 170 is removed from both the proximal and distal portions 152, 154 of the third channel 150, the tube can be fully detached or separated from the sleeve 100, which enables additional functionality. Although conventional intubation can be achieved using the sleeve 100 equipped with a video device as a conventional video laryngoscope, in which case an endotracheal tube is advanced into the oral cavity beneath the sleeve under direct visualization until it is visible on a video monitor connected to the video device, the second tube 170 can be used to facilitate intubation when it has been separated from the sleeve 100 in situ. An example intubation procedure is described below.

Prior to intubation, the sleeve 100 can be prepared for use by inserting a video device into the first channel 112 of the sleeve 100 (in essence forming a video laryngoscope), connecting a fluid (e.g., oxygen) source to the first tube 128, and connecting a suction source to the second tube 170. The blade 104 of the sleeve 100 can be inserted into the oral cavity and advanced to a point at which the vocal cords are clearly visible on the video monitor. Because fluid is driven across the viewing window 116 of the sleeve 100, the viewing window should be clear of material that could otherwise obscure the view of the vocal cords.

Once the sleeve 100 is correctly positioned within the patient airway and a clear view of the vocal cords has been established, the suction source (or a tube that connects the suction source to the second tube 170) can be detached from the second tube, freeing its inner lumen for use as a working channel. An airway-exchange catheter can then be passed through the second tube 170 and advanced into the trachea under video visualization. Next, the second tube 170 can be removed from the proximal and distal portions 152, 154 of the third channel 150 to separate the tube from the sleeve 100. At that point, the second tube 170 can be removed from the patient by withdrawing it over the airway-exchange catheter while simultaneously maintaining the catheter in place within the trachea.

Once the second tube 170 has been removed from both the airway-exchange catheter and the patient, an endotracheal tube can be passed over the catheter and advanced into the trachea using the catheter as a guide under video visualization provided by the video device. Once it is confirmed that the endotracheal tube is properly positioned and secured within the trachea as per protocol, the airway-exchange catheter can be removed from the endotracheal tube and the patient. The sleeve 100 and the video device it contains can then be removed from the patient, the endotracheal tube can be externally secured to the patient, and apparatus, such as a respirator, can be connected to the endotracheal tube. In view of the above description, the separability or detachability of the second tube 170 enables the tube to be used as an introducer that facilitates tracheal intubation.

It is noted that the detachability of the second tube 170 can be utilized in other ways. For example, once the second tube 170 has been used to advance another medical instrument into the patient airway, the tube can be detached from the sleeve 100 and removed from the patient, leaving the sleeve within the airway and the medical instrument within its third channel 150, which had previously been occupied by the tube. Alternatively, once the second tube 170 has been detached from the sleeve 100, the sleeve can be removed from the patient, leaving the tube and whatever medical instrument it contains within the airway.

In cases in which a medical instrument to be introduced into the airway is too large to be passed through the inner lumen of the second tube 170, the tube can be detached from the sleeve 100, either before or after the sleeve has been advanced into the airway, and the third channel 150 can be used as a passage through which the medical instrument can be guided and advanced into the airway. If the second tube 170 is removed from the sleeve 100 prior to insertion of the sleeve into the oral cavity, the medical instrument can have been “preloaded” within the third channel 150 and held in place with the operator's hand as the sleeve is inserted into the cavity and advanced.

Although the above discussion has focused on the removability and separability of the second tube 170, it is noted that the first tube 128 is also removable from the second channel 120 and, therefore, separable from the sleeve 100. In view of that, the first tube 128 could be used for additional purposes similar to those described above in relation to the second tube 170, if desired.

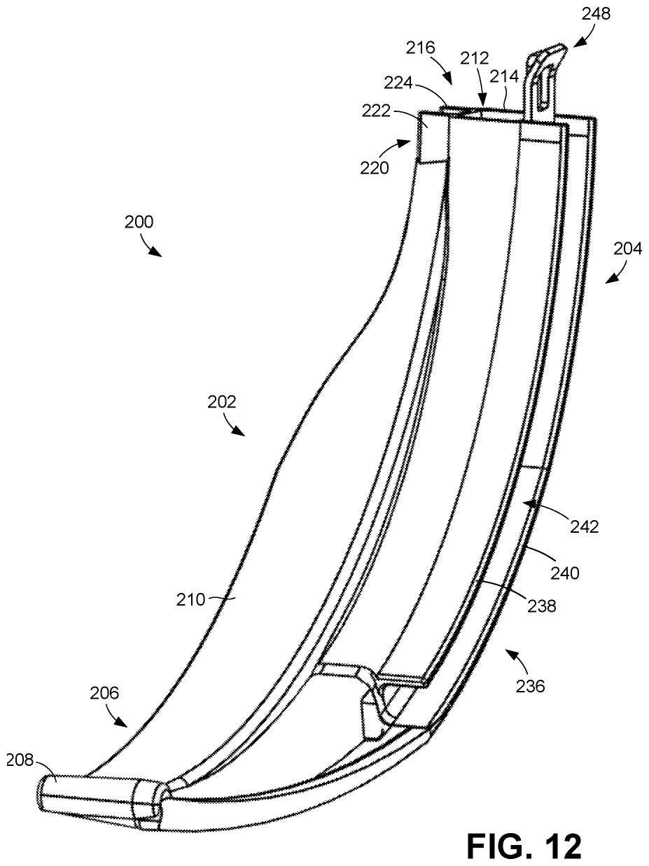

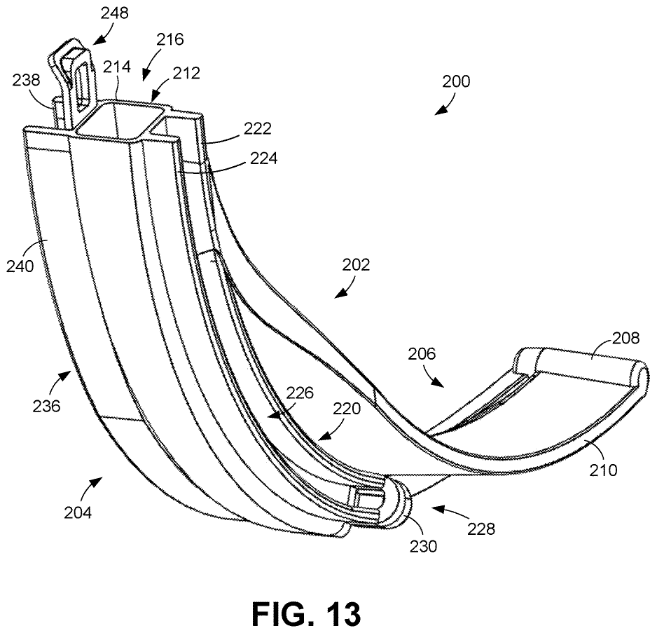

FIGS. 12 and 13 illustrate a third example embodiment of a video laryngoscopy apparatus 200 that includes means for clearing material from a viewing window of the apparatus. As with the apparatus 100 of FIGS. 7 and 8, the apparatus 200 shown in FIGS. 12 and 13 comprises a disposable video laryngoscopy sleeve or sheath that is configured to receive a video device. In this embodiment, however, the apparatus 200 does not include a handle. Instead, as described below, the apparatus 200 comprises an elongated blade that is configured to mount to a video device having its own handle, such as a McGrath™ video laryngoscope. In spite of that difference, the apparatus 200 also may be referred to as a video laryngoscopy sleeve or sheath as it is configured to receive a portion of the video device that extends from the handle of the device. Therefore, for the remainder of the discussion of the video laryngoscopy apparatus 200, the apparatus will be referred to as a sleeve 200. As will be apparent from the disclosure that follows, when a video device is received by the sleeve 200, the resulting combination can be referred to and used as a video laryngoscope.

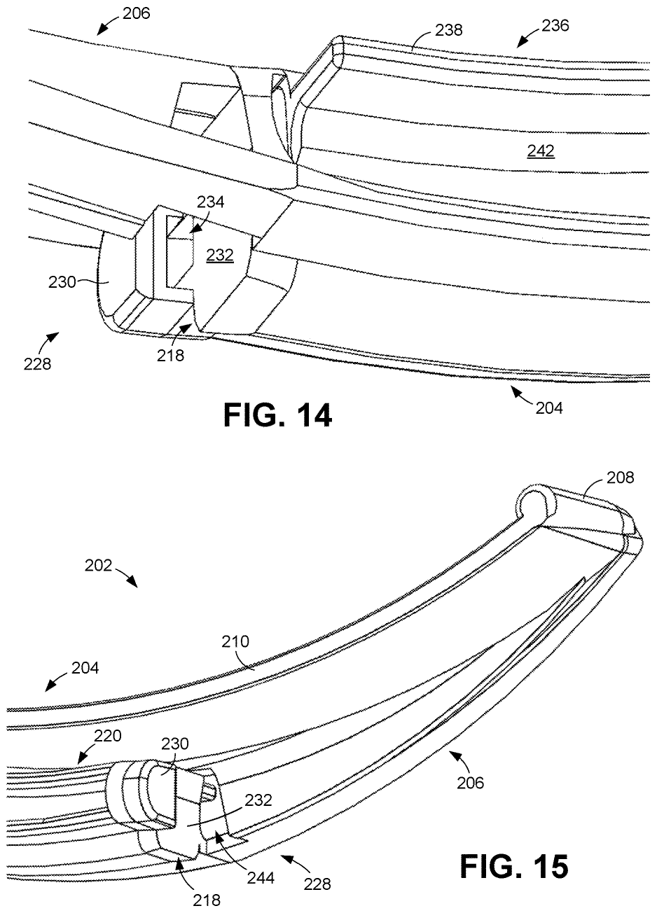

The sleeve 200 shares many similarities with the elongated blade 104 of the sleeve 100 of FIGS. 7 and 8. Accordingly, the sleeve 200 comprises an elongated, curved blade 202 that is configured for insertion through the mouth and oral cavity, and into the throat for the purpose of intubating a patient. In addition, the entire sleeve 200 can be unitarily formed from a single piece of an inexpensive, rigid or semi-rigid, clear material, such as a polymer material like polycarbonate. As is apparent from FIGS. 12 and 13 (as well as FIGS. 14 and 15), the blade 202 includes a relatively thick proximal portion 204 and a relatively thin distal portion 206 that extends from the proximal portion. The distal portion 206 forms a distal tip 208 of the blade 202. As can be appreciated from FIGS. 12 and 13, the distal portion 206 and its distal tip 208 are in part formed by a planar, curved spatula 212 that extends along substantially the entire length of the blade 204.

Extending through the proximal portion 204 of the blade 202 is a relatively large, central, first channel 212 that is configured to receive the video device. As such, the first channel 212 also may be referred to as the video device channel. In some embodiments, the first channel 212 is specifically configured to receive, through a proximal opening 214 of the channel formed at a proximal end 216 of the blade 202, the portion of the video device that extends from the handle of the device such that its camera is positioned at the distal end of the channel in contact with or adjacent to a viewing window 218 (see FIGS. 14 and 15). As is most clearly apparent from FIGS. 13 and 14, the first channel 212 and the viewing window 218 can, in some embodiments, have a generally rectangular cross-section.

With reference to FIG. 13, the sleeve 200 also includes a second channel 220 that forms part of and extends along a first lateral side (the right side from the perspective of the operator) of the proximal portion 204 of the blade 202. As with the second channel 120 of the sleeve 100, the second channel 220 of the sleeve 200 is an elongated, open, external channel whose base is defined by an outer surface of the proximal portion 204 of the blade 202 and whose sides are defined by two spaced, parallel, elongated flanges 222 and 224 that extend generally perpendicularly outward from the outer surface. With that configuration, the outer surface and the flanges 222, 224 define an interior space 226 that is configured to receive and hold a detachable first tube (not shown), and the second channel 220 may be described as a C- or U-shaped channel.

As with the sleeve 200 of FIGS. 7 and 8, the detachable first tube can be positioned within the interior space 226 defined in part by the flanges 222, 224 so that the tube occupies the entire length of the second channel 220 and a distal opening of the tube is positioned at a distal end of the second channel. The first tube can have an outer dimension (e.g., outer diameter) that is slightly greater than the width of the second channel 220 such that the tube is secured within the channel by compressive force and friction, but can be manually removed. In view of that, the second channel also may be referred to as means for releasably securing the first tube. Optionally, one or more releasable locking elements (additional means for releasably securing) can be provided on the proximal portion 204 of the blade 202 to ensure that the first tube is securely held within the second channel 220 unless and until the operator wishes to remove the tube from the channel.

The distal end of the second channel 220 coincides with a distal end 228 of the proximal portion 204 of the blade 202 and is terminated by an end wall 230. The portion of the first tube that does not fit within the second channel 220 can extend outward from the proximal end 216 of the blade 202 for connection to a fluid source. As with the sleeve 100, fluid from the fluid source can be supplied through an inner lumen of the first tube to the distal end 218 of the proximal portion 204 of the blade 202 for use in clearing material from the viewing window 218. In view of that, the first tube also may be referred to as a fluid supply tube, its distal opening also may be referred to as an outlet opening, and the second channel 220 also may be referred to as a fluid supply channel.

FIG. 14 illustrates an underside of a distal end of the blade 202 and, more particularly, the distal end 228 of the proximal portion 204 of the blade. As shown in the figure, the viewing window 218 includes a planar outer surface 230 and forms part of the distal end 218 of the proximal portion 204 of the blade 202 because the viewing window is unitarily formed with the remainder of the blade. On a first lateral side (the right side from the perspective of the operator) of the viewing window 218 is a first fluid passage 234 through which fluid ejected from the distal opening of the first tube can be directed for the purpose of removing material from the outer surface 232 of the viewing window. The fluid ejected by the first tube 228 is forced to pass through the first fluid passage 234 due to the provision of the end wall 230 as there is no other exit through which the fluid can pass.

As the end wall 230 is generally perpendicular to the first fluid passage 234, the end wall causes a generally 90-degree change of direction of the flow of the fluid so that, instead of traveling in a direction generally parallel to the distal portion 204 of the blade 202, the fluid is forced to travel in a direction that is generally parallel to the outer surface 232 of the viewing window 218, similar to the jet of fluid ejected by the nozzle 36 of the sleeve 10. As such, the ejected fluid travels transversely across the viewing window 218 in a direction generally parallel to the outer surface 232 of the viewing window so as to traverse the viewing window from a first lateral side (the right side from the perspective of the operator) to a second lateral side (the left side from the perspective of the operator) of the window. In some embodiments, the first fluid passage 234 can be sized and configured to form a nozzle from which a jet of fluid, such as a gas or a liquid, can be ejected with sufficient force to clear substantially any material that has accumulated on the outer surface 232 of the viewing window 218.

With reference back to FIG. 12, the sleeve 200 further includes a third channel 236 that forms part of and extends along a second lateral side (the left side from the perspective of the operator) of the sleeve. As with the second channel 220, the third channel 236 is an elongated, open, external channel, with the base of the channel being defined by the outer surface of the blade 202 and the sides being defined by two spaced, parallel, elongated flanges 238 an 240 that extend generally perpendicularly outward from the outer surface. With that configuration, the flanges 238, 240 define an interior space 242 configured to receive and secure a detachable second tube (not shown), and the third channel 236 may be described as a C- or U-shaped channel.

The detachable second tube can be positioned with the third channel 236 so that the tube occupies the entire length of the channel and the portion of the second tube that does not fit within the third channel can extend outward from the proximal end 216 of the blade 202 for connection to a suction source. Like the first tube, the second tube can be held within its channel 236 by compressive force and friction and, optionally, one or more releasable locking elements (i.e., means for releasably securing) provided on the proximal portion 204 of the blade 202. Also like the second tube 170 of the sleeve 100, the second tube provided in the third channel 236 can be removed from the channel by simply pulling the tube with enough force to separate the tube from the channel.

When a suction source is connected to the second tube, the second tube can be used to remove at least some of the fluid ejected from the first tube and the material that fluid has removed from the outer surface 232 of the viewing window 218. In such cases, the second tube also may be referred to as the suction tube, its distal opening also may be referred to as an inlet opening, and the third channel 236 also may be referred to as the suction channel. Notably, however, the second tube and the third channel 236 can be used for other purposes, just like the second tube 170 and the third channel 150 of the sleeve 100.

As with the sleeve 100, the distal end of the second tube can be positioned at a distal end of the third channel 236 (which coincides with the distal end 228 of the proximal portion 204 of the blade 202) on a second lateral side (the left side from the perspective of the operator) of the viewing window 218 so that the distal end of the tube is positioned on the opposite side of the viewing window from the first fluid passage 234. In this case, however, the distal end of the tube is positioned behind a second fluid passage 244 formed in the distal portion 206 of the blade 202. With that positioning, the liquid ejected from the first fluid passage 234 and the material the liquid has cleared from the viewing window 218 can pass through the second fluid passage 244 to reach the second tube so that the liquid and material can be removed through the tube by suction force.

Although it comprises no handle, the sleeve 200 can be mounted to a suitable video device, such as a McGrath™ video laryngoscope, with a mounting element 248 provided at the proximal end 216 of the sleeve 200 (see FIGS. 12 and 13). When mounted to the video device, the sleeve 200 can be used in similar manner to the sleeve 100. Accordingly, the second tube, as well as the third channel 236 in which the tube is positioned, can be used for purposes in addition or in exception to applying suction to remove fluid and material accumulated on the viewing window 218. For example, the inner lumen of the second tube can be used as a working channel through which other medical instruments can be passed and positioned within the patient airway. In addition, the second tube can be removed from the third channel 236 and separated from the sleeve 200 for use as a working channel separate from the sleeve and/or for use as an introducer that can facilitate intubation, as described above in relation to the sleeve 100.

Furthermore, in cases in which a medical instrument to be advanced into the patient airway is too large to be passed through the inner lumen of the second tube, the second tube can be detached from the sleeve 300, either before or after the sleeve has been advanced into the airway, and the third channel 236 can be used as a passage or guide through which the medical instrument can be advanced.

Although the above discussion of the sleeve 200 has been focused on the removability and separability of a second (suction) tube from the sleeve, it is noted that the first (fluid supply) tube can, in some embodiments, also be removed from the second channel 220 and, therefore, separated from the sleeve. In view of that, the first tube can be used for additional purposes similar to those described above in relation to the second tube, if desired.

Although the example video laryngoscopy apparatuses disclosed herein have been described as being configured as sleeves or sheaths that receive a separate video device, it is noted that, in other embodiments, the video laryngoscopy apparatuses can include their own integrated cameras, in which case the apparatuses can be referred to as video laryngoscopes. In view of that possibility, the term “video laryngoscopy apparatus” is used broadly herein to identify apparatuses that facilitate video laryngoscopy. Accordingly, that term applies not only to apparatuses for use with video devices, including laryngoscopy sleeves and laryngoscopy blades, but also to video laryngoscopes having their own integrated cameras.

Claimed are:

1. A video laryngoscopy apparatus comprising:
a blade configured for insertion through an oral cavity of a patient;
a clear viewing window provided on the blade that enables the patient's airway to be imaged when a separate video device is received by the video laryngoscopy apparatus; and
a fluid nozzle configured to eject a jet of fluid transversely across the viewing window from a first side of the viewing window to a second, opposite side of the viewing window to drive the material from an outer surface of the viewing window, wherein the jet of fluid is ejected in a direction generally parallel to the outer surface of the viewing window.

2. The video laryngoscopy apparatus of claim 1, wherein the video laryngoscopy apparatus comprises a disposable sleeve configured to receive the video device.

3. The video laryngoscopy apparatus of claim 1, further comprising a fluid supply channel or tube configured to supply the fluid to the fluid nozzle.

4. The video laryngoscopy apparatus of claim 1, further comprising a suction channel or tube configured to collect and remove at least some of the ejected fluid and cleared material.

5. The video laryngoscopy apparatus of claim 1, wherein the fluid nozzle is positioned adjacent to a side of the clear viewing window and wherein the viewing window and the fluid nozzle are both unitarily formed from a single piece of material such that the fluid nozzle is integrated with the viewing window.

6. The video laryngoscopy apparatus of claim 1, wherein the fluid nozzle comprises a fluid passage unitarily formed by a single piece of material from which the entirety of the video laryngoscopy apparatus is made.

7. A video laryngoscopy sleeve configured to receive a separate video device, the video laryngoscopy sleeve comprising:
a blade configured for insertion through an oral cavity of a patient;
a clear viewing window provided on the blade that enables the patient's airway to be imaged;
means for clearing material from the clear viewing window that could obscure a view of the airway, the means being configured to eject fluid across an outer surface of the viewing window to drive the material from the viewing window;
a flexible tube having a proximal opening, a distal opening, and a lumen that extends between the openings, the flexible tube being removably attached to the blade; and
means for releasably securing the flexible tube to the sleeve such that the distal opening of the tube is positioned adjacent to the clear viewing window;
wherein the flexible tube can be partially or fully separated from the blade to enable the tube to be used to advance one or more other medical instruments into the patient's airway.

8. The video laryngoscopy sleeve of claim 7, wherein the blade is curved.

9. The video laryngoscopy sleeve of claim 7, wherein the blade comprises a relatively thick proximal portion and a relatively thin distal portion.

10. The video laryngoscopy sleeve of claim 9, wherein the proximal portion of the blade is configured to receive at least part of the video device in a manner in which a camera of the video device is positioned adjacent to a distal end of the proximal portion at which the clear viewing window is located.

11. The video laryngoscopy sleeve of claim 7, wherein the means for clearing material comprise a fluid nozzle adjacent to the clear viewing window that is configured to drive the material from the outer surface of the viewing window.

12. The video laryngoscopy sleeve of claim 11, wherein the fluid nozzle is configured to eject a jet of fluid transversely across the viewing window from a first side of the viewing window to a second, opposite side of the viewing window in a direction generally parallel to the outer surface of the viewing window.

13. The video laryngoscopy sleeve of claim 11, wherein the flexible tube is a fluid supply tube that is configured to supply fluid to the fluid nozzle and wherein the means for releasably securing comprises an elongated, open, external channel that extends along an outer side of the blade in which the tube is removably secured.

14. The video laryngoscopy sleeve of claim 13, wherein the external channel is at least partly defined by two parallel, elongated flanges that extend outward from and along a length of the blade, wherein the flanges define an elongated interior space in which the fluid supply tube can be held.

15. The video laryngoscopy sleeve of claim 14, wherein the means for releasably securing further comprise releasable locking elements provided on the video laryngoscopy sleeve.

16. The video laryngoscopy sleeve of claim 11, wherein the flexible tube is a suction tube that is configured to collect and remove at least some of the ejected fluid and cleared material and wherein the means for releasably securing comprises an elongated, open, external channel that extends along an outer side of the blade in which the suction tube is removably secured.

17. The video laryngoscopy sleeve of claim 16, wherein the external channel is at least partly defined by two parallel, elongated flanges that extend outward from and along a length of the blade, wherein the flanges define an elongated interior space in which the suction tube can be held.

18. The video laryngoscopy sleeve of claim 17, wherein the means for releasably securing further comprise releasable locking elements provided on the video laryngoscopy sleeve.

19. The video laryngoscopy sleeve of claim 11, further comprising a handle from which the blade extends, the handle being configured for gripping by an operator and also being configured to receive at least part of the video device.

20. A method for performing a video laryngoscopy procedure, the method comprising:
inserting a video device into a disposable video laryngoscope sleeve;
connecting a fluid source to a fluid supply tube that is removably attached to the video laryngoscope sleeve;
connecting a suction source to a suction tube that is removably attached to the video laryngoscope sleeve;
inserting a blade of the video laryngoscope sleeve into an oral cavity of a patient and advancing the blade into an airway of the patient to a point at which the patient's vocal cords are visible on a video monitor connected to the video device, wherein material that is deposited on a viewing window of the video laryngoscope sleeve during the inserting and advancing is cleared by fluid supplied by the fluid supply tube and is removed by the suction tube;
detaching the suction source from the suction tube; and
partially or fully detaching the suction tube from the video laryngoscope sleeve.

21. The method of claim 20, wherein detaching the suction tube comprises removing the suction tube from an elongated, open, exterior channel that extends along an outer side of the video laryngoscope sleeve.

22. The method of claim 21, further comprising, either before or after detaching the suction tube, advancing a first separate medical instrument through an inner lumen the suction tube and into the patient's airway.

23. The method of claim 22, wherein partially or fully detaching the suction tube comprises fully detaching the suction tube and further comprising removing the suction tube from the patient while leaving the first separate medical instrument in place within the patient's airway.

24. The method of claim 23, wherein advancing the first separate medical instrument comprises advancing the first separate medical instrument into a trachea of the patient and further comprising advancing a second separate medical instrument over the first separate medical instrument and into the trachea.

25. The method of claim 24, wherein the first separate medical instrument is an airway-exchange catheter and the second separate medical instrument is an endotracheal tube.
